(19) United States
(12) Patent Application Publication
(10) Pub. No.: US 2003/0224034 A1
(43) Pub. Date: Dec. 4, 2003

(54) Personal care article and method for inhibiting attachment of yeast to skin

(76) Inventor: David W. Koenig, Menasha, WI (US)

Correspondence Address: PAULEY PETERSEN KINNE & ERICKSON, 2800 WEST HIGGINS ROAD, SUITE 365, HOFFMAN ESTATES, IL, 60195, US

(21) Appl. No.: 10/159,253

(22) Filed: May 31, 2002

(51) Int. Cl. A61F 13/15; A61F 13/20; A61K 9/70

(52) U.S. Cl. 424/443; 604/375

(57) ABSTRACT

A personal care article including an isoprenoid compound, and a method of inhibiting attachment of pathogenic fungi to skin using an isoprenoid compound in combination with a personal care article. The isopreniod compound may be farnesol, which is particularly effective against the pathogenic yeast Candida albicans.

### BACKGROUND OF THE INVENTION

This invention is directed to a personal care article including an isoprenoid compound, such as farnesol, and a method of inhibiting attachment of yeast to skin using an isoprenoid compound, such as farnesol.

The growth and attachment of the pathogenic yeast Candida albicans on human skin has been associated with numerous ailments such as thrush in infants, diaper rash in infants, and urinary/vaginal infections in adult females. Other fungi that adhere to human skin and subsequently grow, causing ailments, include Mallessia, Tricophyton, Epidermophyton, Scytalidium, Fusarium, Acremonium, Aspergillus, Scopulariopsis, and Pityrosporum.

Adherence to epithelial cells is the first step in colonization by Candida and other fungi, followed by establishment of mucocutaneous infection. Similarly, adherence to intravascular structures is considered to be a critical step in the infection of blood-borne fungi to target organs. Optimal therapy in treating Candida and other fungi requires strategies to increase host resistance to yeast or other fungal infection, combined with the use of antifungal agents. Antifungal agents destroy or inhibit the growth of fungi, thereby fighting fungal infections.

If pathogenic fungi could be prevented from adhering to skin or mucous membranes in the first place, a potential fungal infection would be prevented from manifesting on or beneath the skin, and there would be no need to destroy or distort the growth of the fungi.

There is thus a need or desire for a treatment that inhibits yeast or fungal attachment to skin and/or mucous membranes to prevent fungal infections from occurring.

### SUMMARY OF THE INVENTION

In response to the discussed difficulties and problems encountered in the prior art, a new method of preventing fungal infections has been discovered. The principles of the present invention may be applied to any of a number of personal care product applications, such as personal care absorbent garments, feminine care articles, health care articles, pre-moistened wipes, absorbent wipes, bath tissue, facial tissue, lotions, and creams.

Farnesol, which is an isoprenoid compound, has been found to have an inhibitory effect on the attachment of pathogenic yeast, and other pathogenic fungi, to skin. Such pathogenic fungi may include Candida albicans, Mallessia, Tricophyton, Epidermophyton, Scytalidium, Fusarium, Acremonium, Aspergillus, Scopulariopsis, and Pityrosporum. By incorporating an effective amount of farnesol or other isoprenoid compound, such as atlantol, cedrol, (−)alpha-bisabolol spathulenol, citronellol, geraniol, borneol, cedrol:borneol, or trans-pinocarveol, into a skin-contacting surface of a personal care article, the resulting article acts to prevent the attachment of pathogenic yeast or other pathogenic fungi to the wearer's skin during use. The isoprenoid compound can be applied to the article in solution at a concentration of between about 0.001% and about 2% by weight of the solution.

The skin-contacting surface of the personal care article may be a nonwoven web or any other suitable substrate to which the isoprenoid compound may be applied. The isoprenoid compound may be applied to the article as a sprayed-on additive, by soaking the article in a solution of the isoprenoid compound, by incorporating the isoprenoid compound into a melt from which the article is made, or by any other suitable method. The isoprenoid compound may be contained within a vehicle used to deliver the isoprenoid from the article to a wearer's skin. Examples of suitable vehicles include lotions, emulsions, creams, gels, aqueous vehicles, encapsulation, microencapsulation, and coating of nanoparticles. In another embodiment of the invention, the skin-contacting surface of the article may include numerous cavities and the isoprenoid compound may be inserted into and stored within the cavities until transferred to the wearer's skin.

Farnesol is derived from the essential oils of various plants, including orange blossom, rose, jasmine, and linden flowers. Plant extracts including these essential oils may be applied to the skin-contacting surface of the personal care article as a manner of incorporating farnesol into the article.

In addition to the isoprenoid compound, the article of the invention may also include compositions that enhance and/or target the delivery of the isoprenoid compound to the wearer's skin, such as petrolatum, alcohols, glycerols, waxes, or other hydrophobic compounds.

With the foregoing in mind, it is a feature and advantage of the invention to provide a personal care article including an isoprenoid compound, and a method of inhibiting attachment of pathogenic fungi to skin using an isoprenoid compound in combination with a personal care article.

### BRIEF DESCRIPTION OF THE DRAWINGS

FIG. 1 representatively shows a partially cutaway, top plan view of a personal care article, namely a personal care absorbent garment, according to one embodiment of the invention.

FIG. 2 representatively shows a perspective view of a personal care article, namely a feminine care product, according to one embodiment of the invention.

FIG. 3 representatively shows a top plan view of a personal care article, namely a medical care article, according to one embodiment of the invention.

FIG. 4 representatively shows a perspective view of a personal care article, namely an absorbent wipe, according to one embodiment of the invention.

### DEFINITIONS

Within the context of this specification, each term or phrase below will include the following meaning or meanings.

“Applied” refers to the contacting, incorporating, joining, adhering, attaching, connecting, bonding, or the like, of at least one element to another element. An element will be considered to be applied to another element when the elements are applied directly to one another or indirectly to one another, such as when each is directly applied to intermediate elements.

“Disposable garment” includes garments which are typically disposed of after 1-5 uses.

“Feminine care article” includes tampons, feminine care pads, and the like.

“Health care article” includes medical care articles, dental care articles, veterinary care articles, bandages, wound dressings, and the like.

“Meltblown fiber” refers to fibers formed by extruding a molten thermoplastic material through a plurality of fine, usually circular, die capillaries as molten threads or filaments into converging high velocity gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such processes are known in the art. Meltblown fibers are microfibers which may be continuous or discontinuous, are generally smaller than about 0.6 denier, and are generally self bonding when deposited onto a collecting surface.

“Nonwoven” and“nonwoven web” refer to materials and webs of material having a structure of individual fibers or filaments which are interlaid, but not in an identifiable manner as in a knitted fabric. The terms“fiber” and “filament” are used herein interchangeably. Nonwoven fabrics or webs have been formed from many processes such as, for example, meltblowing processes, spunbonding processes, air laying processes, and bonded carded web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

“Personal care absorbent garment” includes diapers, diaper pants, training pants, absorbent underpants, swim wear, incontinence products, and the like. Personal care absorbent garments are typically disposable.

“Personal care article” includes personal care absorbent garments, feminine care articles, health care articles, pre-moistened wipes, absorbent wipes, bath tissue, facial tissue, lotions, and cream, and the like.

“Spunbond fiber” refers to small diameter fibers which are formed by extruding molten thermoplastic material as filaments from a plurality of fine capillaries of a spinnerette having a circular or other configuration, with the diameter of the extruded filaments then being rapidly reduced, as known in the art. Spunbond fibers are quenched and generally not tacky when they are deposited onto a collecting surface. Spunbond fibers are generally continuous and often have average deniers larger than about 0.3, more particularly, between about 0.6 and 10.

These terms may be defined with additional language in the remaining portions of the specification.

### DETAILED DESCRIPTION OF THE PRESENTLY PREFERRED EMBODIMENTS

The present invention is directed to a personal care article including an isoprenoid compound, and a method of inhibiting attachment of pathogenic fungi to skin using an isoprenoid compound in combination with a personal care article.

Isoprenoid compounds have been found to inhibit attachment of pathogenic fungi to skin and/or mucous membranes. By inhibiting the attachment of yeast or other fungi to the skin and/or mucous membranes, an infection process initiated by the yeast or other fungus cannot proceed.

Isoprenoids are a class of largely hydrophobic or nonpolar compounds related by being constructed biosynthetically from five-carbon units. Farnesol (3,7,11-trimethyl-2,6,10-dodecatrien-1-ol) is an isoprenoid compound, which in its activated form (esterified to pyrophosphate) is known as farnesyl diphosphate, and is an intermediate in cholesterol biosynthesis. Farnesol has been shown to be a very effective and skin-compatible substance against body odor and has been used in deodorants, foot care products, and anti-dandruff shampoos. Its deodorant properties are derived through inhibition of gram-positive bacteria growth that is associated in the conversion of sweat into unpleasant odors. Farnesol is present in nature in the essential oils of orange blossom, rose, jasmine, and linden flowers. Farnesol has been recently implicated in the control of dimorphism in the pathogenic yeast Candida albicans. Farnesol has been found to be useful in the present invention because of its ability to inhibit attachment of C. albicans, as well as other pathogenic fungi, to human skin. Other isoprenoid compounds believed to have this anti-attachment activity include other terpene alcohols, namely atlantol, cedrol, (−)alpha-bisabolol spathulenol, citronellol, geraniol, borneol, cedrol:borneol, and trans-pinocarveol, as well as sesquiterpenes, such as chamazulene, caryophyllene, cadinene, elemene lauradiol chamazulene, dihydrochamazulene I & II, bisabolenes, farnesene a+b caryophyllenes, a+b humulene, a-amorphene, a-muurrolene, calamene, calacorene, alpha-cedrene, and cadinene. Other terpene-like compounds that are believed to have this anti-attachment activity include the following sesquiterpenoids: Chamazulene; Guaiazulene; Bazzanene; Muurolene; Isolongifolene; delta-Cadinene; beta-Humulene; beta-Elemene; beta-Bisabolene; Longifolene; alpha-Guaiene; Selinene (alpha-isomer); Aromadendrene; Acoradin; Germacrene D; 4-(1,5-Dimethyl-hexa-1,3-dienyl)-1-methyl-cyclohexene; beta-Caryophyllene; Aromadendrene; Drimane-7,9(11)-diene; Ylangene; Santalene (alpha-); Thujopsene; Alloaromadendrene; Cedrene (alpha-isomer); Humulene; Dihydrothujopsene; Pentamethyloctalin; Linderazulene; Dihydrolinderazulene; Lactaroviolin; Deterrol; 7-Hydroxy-cadalin; Debromolaurinterol; ar-Turmerone; 7-Hydroxy-3,4-dihydro-cadalin; 2-Methyl-6-p-tolyl-hept-3-en-2-ol; Thujopsenal; Radulol; Sinensal (unknown isomer); 2-Methyl-6-p-tolyl-hept-2-en-4-ol; (1)10-Aristolen-2-one; alpha- and beta-Santalol; Caryophyllene oxide; Cedrenol; Farnesal; Cedral; Piconia; Caryophyllodienol; Ylangenol; Thujopsenol; Humulene-2,3-epoxide; Bazzanenol; Partheniol; Vetiverol; Allospathulenol; 3-Acetoxy-thujopsene; Spathulenol; 8,12,12-Trimethyl-4-oxa-tricyclo[6.4.0.01,3]dodecan-5-one; 1-Hydroxymethylene-5,5,8a-trimethyl-octahydro-naphthalen-2-one; Epicubenol; Patchouli alcohol; Widdrol; 1,2,3a,6-Tetramethyl-decahydro-cyclopenta[c]pentalen-2-ol; Cedrol; alpha-Eudesmol; Epiglobulol; Globulol; Farnesol; 2-cis-6-trans-Farnesol; alpha-Bisabolol; trans-Nerolidol; Nerolidol; 2-trans-6-cis-Farnesol; Palustrol; Ledol; delta-Cadinol; Elemol; alpha-Cadinol; 2-trans-6-trans-Farnesol; 2-cis-6-cis-Farnesol; Thujopsan-3-ol; (+)-T-Cadinol; Viridiflorol; Prostantherol; T-Muurolol; 2-alpha-Hydroxymethyl-2,4alpha,8,8-tetramethyl-delta8a-octalin; (6E)-2,3-Dihydrofarnesol; trans-Dihydronerolidol; cis-Dihydronerolidol; Tetrahydronerolidol; Hexahydrofarnesol; Hexahydronerolidol; Axisonitrile-3;Mexicanin E; 6-(2-Hydroxy-4-methyl-phenyl)-2-methyl-hept-2-en-4-one; (+)-Isovelleral isomer 2; 8-Ketoangenal; Isoisovelleral; Exovelleral A; Exovelleral B; 6-(3-Hydroxy-4-methyl-phenyl)-2-methyl-hept-2-en-4-one; Collybial; ar-5-Hydroxyturmerone; (+)-Isovelleral; Costunolide; c8-Ketocopaenal; Velleral; (−)-Isovelleral; Isovellerol; Epipolygodial; Polygodial; Hinokiic acid; 8-Ketoangenol; Vellerol; 11betaH, 13-Dihydrocostunolide; Sclerocarpic acid; 8-Ketocopaenol; Sponge sesquiterpene; 11-Dihydro-polygodial; 11-Dihydro-9-epipolygodial; Davanone; Vellerdiol; 7-Deacetoxyolepupuane; 3,6-Epoxydioxy-bisabola-1,10-diene; 6,6,9a-Trimethyl-decahydro-naphtho[1,2-c]furan-1-one; Farnesyl methyl ether; Cedramber; 7-Hydroxy-6,11-cyclofarnes-3(15)-en-2-one; Debneyol; Shiromool; Aromadendrane-7-alpha,11beta-diol; 8-Hydroxy-elemol; Alloaromadendran-7alpha,11beta-diol; Alloaromadendran-7beta,11beta-diol; alpha-Bisabolol oxide (A-form); Aromadendrane-7beta,11beta-diol; 4-Isopropyl-1,6-dimethyl-1,2,3,4,4a,7,8,8a-octahydro-naphthalene-1-thiol; Lettucenin A; 3-(1-Hydroxy-4,8,8-trimethyl-spiro[2.5]oct-4-yl)-propionic acid; Encelin; Yomogin; 3,7,8-Trimethyl-9,9a-dihydro-5h,8h-azuleno[6,5-b]furan-2,6-dione; Deacetoxymatricarin; Radulone A; 3,7,8-Trimethyl-4a,5,9,9a-tetrahydro-4h,8h-azuleno[6,5-b]furan-2,6-dione; Radulactone; Acetylcedren; Taurin; Exomerulidal; Eupatolide; Merulidial; 9-Hydroxyisovelleral; 9-Hydroxy-Isoisovelleral; Parthenolide; Tayunin; 1,10-Epoxycostunolide; Dermatolactone; Muzigadial; Reynosin; 11betaH, 13-Dihydroparthenolide; 1,10-Epoxy-11betaH,13-dihydrocostunolide; Warburganal; Santamarine; 11betaH,13-Dihydrosantamarine; 11betaH,13-Dihydroreynosin; 1-Methoxy-6,6,9a-trimethyl-dodecahydro-naphtho[1,2-c]furan; 3-Isopropyl-6,10-dimethyl- 11 -oxa-bicyclo[8.1.0]undec-6-ene-2,8-diol; Reynosin triol derivative; 4beta,5alpha-Epoxy-7alphaH-germacr-10(14)-ene-1beta,6beta-diol; Santamarine triol derivative; 7-(1-Hydroxy-1-methyl-ethyl)-1,4a-dimethyl-decahydro-naphthalene-1,8-diol; 11,13-Dihydroreynosin triol derivative; 5-(1-Hydroxy-1-methyl-ethyl)-2-methyl-8-methylene-cyclodecane-1,6-diol; Helenalin; 4a,8-Dimethyl-4,7-oxo-3-methylene-decahydro-azuleno[6,5-b]furan-2,5-dione; 4a,8-Dimethyl-7,7a-oxo-3-methylene-decahydro-azuleno[6,5-b]furan-2,5-dione; Farinosin; Marasmic acid; Cedrenyl acetate; 4-Isopropyl-5-isothiocyanato-1,5-dimethyl-1,2,3,4,4a,5,6,7-octahydro-naphthalene; 3beta,8beta-Dihydroxy-11alphaH-guaia-4(15),10(14)-diene-12,6alpha-olide; Plenolin; 4-Hydroxy-4a,8-dimethyl-3-methylene-decahydro-azuleno[6,5-b]furan-2,5-dione; Vulgarin; 4,9-Dihydroxy-6,10-dimethyl-3-methylene-3a,4,5,8,9,11a-hexahydro-3h-cyclodeca[b]furan-2-one; Michelenolide; Ridentin; 5,6-Diformyl-1,4a-dimethyl-1,2,3,4,4a,5,8,8a-octahydro-naphthalene-1-carboxylic Acid; 9-alpha-Hydroxy-merulidial; Lactardial; 5a,6-Dihydroxy-4,4,6a-trimethyl-3,4,5,5a,6,6a-hexahydro-1h-cyclopropa[f]indene-1a,2-dicarbaldehyde; Neopentyl acetate; Cedryl acetate; Guaiyl acetate; Vetiveryl acetate; Farnesyl acetate; Rosa rugosa aldehyde; Leptospermone; 1,10-Epoxy-11betaH,13-dihydroparthenolide; 4-Hydroxy-3,4a,8-trimethyl-decahydro-azuleno[6,5-b]furan-2,5-dione; 1-Hydroxy-6,9a-dimethyl-1,3,5,5a,6,7,8,9,9a,9b-decahydro-naphtho[1,2-c]furan-6-carboxylic acid; Mukadial; Methyl marasmate; 9-Hydroxymarasmic acid; 11-Dihydroxy-drim-8-ene-12,13-dioic acid 13-methyl ester 11 12-lactone; 7-Hydroxy-2,6,7a-trimethyl-decahydro-1,4-dioxa-cyclopenta[f]cyclopropa[a]azulene-5,8-dione; Methyl 2-oxo-3,5a,8-trimethylperhydroindeno[4,5-b]furan-7-carboxylate; 8-Acetoxy-elemol; Hymenovin; 1-Methoxy-6,6,9a-trimethyl-decahydro-naphtho[1,2-c]furan-3,3a-diol; Plumericin; Micanolide; Acetyl-merulidial; Acetyl-isomerulidial; Dihydromicanolide; Acetic acid, 6,6,9a-trimethyl-3-oxo-1,3,5,5a,6,7,8,9,9a,9b-decahydro-naphtho[1,2-c]furan-1-yl ester; Acetic acid, 6,6,9a-trimethyl-3-oxo-1,3,4,5,5a,6,7,8,9,9a-decahydro-naphtho[1,2-c]furan- 1-yl ester; Allolaurinterol; 10-Hydroxy-2-isopropyl-5-methyl-11,12-dioxa-tricyclo[5.3.2.0-1,5]-8-dodecene-8-carboxylic acid, methyl ester; Helenalin acetate; Acetic acid, 4a,8-dimethyl-3-methylene-2,5-dioxo-2,3,3a,4,4a,5,7a,8,9,9a-decahydro-azuleno[6,5-b]furan-4-yl ester; 11,13-Dihydrohelenalin acetate; Sesquiterpene lactone IV; 9-alpha-Hydroxyacetylmerulidial; Tenulin; Carolenalin monoacetate; Acetic acid, 1-acetoxymethyl-5,5,8a-trimethyl-decahydro-naphthalen-2-yl ester; alpha-Arteether; beta-Arteether; Avarol; 3-Methyl-but-2-enoic acid, 4a,8-dimethyl-3-methylene-2,5-dioxo-2,3,3a,4,4a,5,7a,8,9,9a-decahydro-azuleno[6,5-b]furan-4-yl ester; Savigraviolide A; 3-O-Deacetyl-9-O-acetylsavigraviolide A; Acetic acid, 1-acetoxymethyl-5,5,8a-trimethyl-decahydro-naphthalen-2-ylmethyl ester; Aplysistatin; Tamaulipin A angelate; Deoxyelephantopin; 6-O-Methacrylplenolin; 6-O-Isobutyroylplenolin; 2-Methyl-but-2-enoic acid, 1-hydroxy-7-isopropyl-1,4a-dimethyl-6-oxo-1,2,3,4,4a,5,6,8a-octahydro-naphthalen-2-yl ester; Sesquiterpene lactone I; 6beta-Hydroxyaplysistatin; Sesquiterpene lactone III; 6-O-Angeloylplenolin; 3-Methyl-butyric acid, 4a,8-dimethyl-3-methylene-2,5-dioxo-2,3,3a,4,4a,5,7a,8,9,9a-decahydro-azuleno[6,5-b]furan-4-yl ester; Sesquiterpene lactone II; Florilenalin diacetate; Acetic acid, 4-acetoxy-6,10-dimethyl-3-methylene-2-oxo-2,3,3a,4,5,8,9,11a-octahydro-cyclodeca[b]furan-9-yl ester; 2-Methyl-but-2-enoic acid, 1-hydroxy-7-(1-hydroxy-1-methyl-ethyl)-1,4a-dimethyl-6-oxo-1,2,3,4,4a,5,6,8a-octahydro-naphthalen-2-yl ester; 2,3-Dimethyl-oxirane-2-carboxylic acid, 1-hydroxy-7-isopropyl-1,4a-dimethyl-6-oxo-1,2,3,4,4a,5,6,8a-octahydro-naphthalen-2-yl ester; 2,3-Dimethyl-oxirane-2-carboxylic acid, 1-hydroxy-7-isopropylidene-1,4a-dimethyl-6-oxo-decahydro-naphthalen-2-yl ester; 5-epi-Isospongiaquinone; 9-O-Acetylsavigraviolide A; 2,3-Dimethyl-oxirane-2-carboxylic acid, 1-hydroxy-7-(1-hydroxy-1-methyl-ethyl)-1,4a-dimethyl-6-oxo-1,2,3,4,4a,5,6,8a-octahydro-naphthalen-2-yl ester; 14-o-Hydroxycinnamoyl-dauc-4,8-diene; 5-epi-Homoisospongiaquinone; (5-Hydroxymethyl-5,8a-dimethyl-2-methylene-decahydro-naphthalen-1-ylmethoxy)-(2-oxo-tetrahydro-furan-3-yl)-acetic acid; Pilatin; 2,3-Dimethyl-oxirane-2-carboxylic acid, 1-acetoxy-7-isopropylidene-1,4a-dimethyl-6-oxo-decahydro-naphthalen-2-yl ester; 2,3-Dimethyl-oxirane-2-carboxylic acid, 1-acetoxy-7-isopropyl-1,4a-dimethyl-6-oxo-1,2,3,4,4a,5,6,8a-octahydro-naphthalen-2-yl ester; (5-Hydroxymethyl-5,8a-dimethyl-2-methylene-decahydro-naphthalen-1-ylmethoxy)-(2-oxo-tetrahydro-furan-3-yl)-acetic acid, methyl ester; Chromolaenide, 4-Hydroxy-2-methyl-but-2-enoic acid, 9-acetoxy-3,6,10-trimethyl-2-oxo-2,3,3a,4,5,8,9,11a-octahydro-cyclodeca[b]furan-4-yl ester; 2,3-Dimethyl-oxirane-2-carboxylic acid, 1-acetoxy-7-(1-hydroxy-1-methyl-ethyl)-1,4a-dimethyl-6-oxo-1,2,3,4,4a,5,6,8a-octahydro-naphthalen-2-yl ester; Judeol; O-Methylmelleolide; PSF-D; Chromolaenide acetate; PSF-B; PSF-A; 7-Isopropenyl-4-methyl-6,7-dihydro-azulen-1-yl Octadecanoate; and 2beta-Acetoxy-4alpha-chloro-1beta,8-diangeloyloxy-3beta,10-dihydroxy-11-methoxy-bisabol-7(14)-ene

In addition to Candida albicans, other types of pathogenic fungi that can be targeted by isoprenoid compounds include but are not limited to, Mallessia, Tricophyton, Epidermophyton, Scytalidium, Fusarium, Acremonium, Aspergillus, Scopulariopsis, and Pityrosporum.

In one embodiment of the invention, a method of inhibiting pathogenic fungal attachment to skin is carried out by applying an effective amount of an isoprenoid compound to a personal care article, particularly a skin-contacting surface of the personal care article. Another embodiment of the invention is directed to the personal care article with the isoprenoid compound applied thereto in an amount effective to inhibit pathogenic fungi attachment to a wearer's skin.

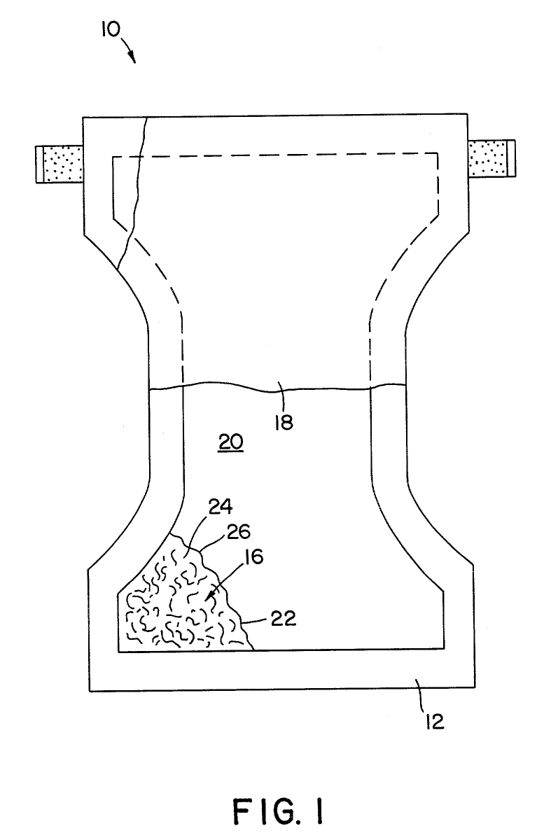

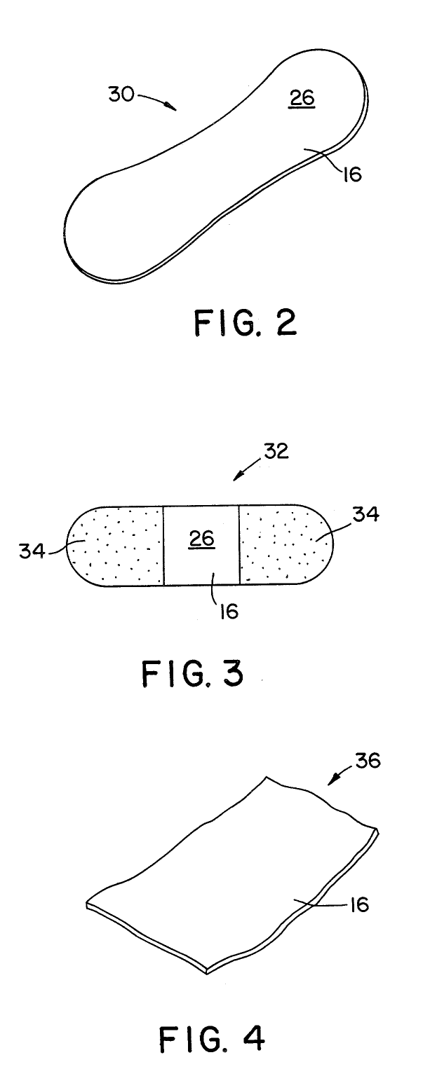

Suitable personal care articles that may be used in accordance with the invention include products that are intimately involved in the cleaning and/or containment of bodily fluids, detritus spills, and/or surfaces contaminated with microorganisms. Such suitable personal care articles include, but are not limited to, personal care absorbent garments, such as diapers, diaper pants, training pants, absorbent underpants, swimwear, and incontinence garments, with an example of a personal care absorbent article shown in FIG. 1. Other suitable personal care articles include feminine care articles, such as tampons and feminine care pads, with an example of a feminine care article shown in FIG. 2. Other suitable personal care articles include health care articles, such as medical care articles, dental care articles, veterinary care articles, bandages, and wound dressings, with an example of a health care article shown in FIG. 3. Other suitable personal care articles include pre-moistened wipes, absorbent wipes, bath tissue, facial tissue, lotions, and creams, with an example of this group shown in FIG. 4.

Isoprenoid compounds that stop yeast adherence to the skin can be incorporated into any of the listed personal care articles or other suitable personal care articles, such that the isoprenoid compound is transferred to the uro-genital region, or other potentially contaminated area of a wearer's body, eliminating the pathogenic fungi from the region, thus reducing or eliminating serious infections. Other problem fungi on the skin, nails, and hair can be similarly controlled using the anti-adherence technology of the invention. Furthermore, the anti-adherence technology of the invention with respect to isoprenoid compounds may be useful in ocular, vaginal, nasal, respiratory, and/or oral health care applications.

Nonwoven webs are particularly suitable materials for the substrate, which can be used to form a skin-contacting surface of a number of personal care articles. For example, the substrate can be composed of a meltblown or spunbonded web of polyolefin fibers. The substrate can also be a bonded-carded web composed of natural and/or synthetic fibers.

Isoprenoid compounds can be applied to the personal care article substrate in a number of different ways. For example, the isoprenoid compound can be incorporated into the nonwoven substrate or other type of substrate as a sprayed-on additive, or may be incorporated into a melt from which the substrate is produced. As another example, the personal care article, or at least the skin-contacting surface of the article, can be coated with the isoprenoid compound, by slot coating, printing (such as flexographic printing), coating (such as gravure coating), extrusion, or combinations of any of these methods, such as spraying the isoprenoid solution on a rotating surface, then transferring the solution to the skin-contacting surface of the personal care article.

The manner of applying the isoprenoid composition to the personal care article should be such that the article does not become saturated with the composition. If the article becomes saturated with the composition, the fluid permeability of certain layers of the article may be reduced or blocked. However, it may be beneficial to saturate certain types of personal care articles with the isoprenoid compositions. For example, the isopreniod compound can be incorporated into a solution, such as a cleansing solution, in which the substrate, such as an absorbent wipe, can be soaked.

As used herein, the term“skin-contacting surface” refers to materials that are both typically and less frequently in contact with a wearer's skin. Examples of suitable materials from which the“skin-contacting surface” may be made include, but are not limited to, materials such as body side liner, elastic material, tissue, intake and distribution material, absorbent material, including, but not limited to, coform, woven and nonwoven materials, back sheet liner material, or any other material known in the art that is or can be used in the construction of personal care articles, such as personal care absorbent garments, feminine care articles, health care articles, pre-moistened wipes, absorbent wipes, bath tissue, and facial tissue. The skin-contacting surface material of the invention can be a single layer or multiple layers.

The isoprenoid compound can be applied to a specific portion or component of the personal care article or to the entire surface of the article that comes into contact with the wearer's skin during use of the article, as long as at least a portion of the skin-contacting surface of the article is treated with the isoprenoid compound.

The amount of isoprenoid compound applied to the article can be routinely determined given the present disclosure, provided that a sufficient quantity is used to produce an anti-attachment effect of fungi to skin. As shown in the Example below, farnesol at a concentration of 2% is able to effectively inhibit the attachment of yeast to skin. More particularly, the isoprenoid compound can be applied to the personal care article substrate in solution at a concentration of between about 0.001% and about 2%, or between about 0.001% and about 0.1%, or between about 0.001% and about 0.01%, by weight of the solution.

In addition, the isoprenoid compound can be applied in varying concentrations or deposition amounts on the skin-contacting surface of the article or portion thereof. The isoprenoid compound is applied such that the isoprenoid can be delivered via contact with the user's skin during the use of the article. The isoprenoid compound can be applied after the skin-contacting material has been incorporated into the article or prior to incorporating the skin-contacting material into the article. The phrase“effective amount” of the isoprenoid compound, or of farnesol, is understood to mean an amount of the isoprenoid compound, or of farnesol in particular, which, when applied to the skin-contacting surface of the article, will be effective in inhibiting attachment of yeast or other fungi to the wearer's skin.

In one embodiment of the invention, the substrate may include degradable hollow fibers or other structures having cavities, and the isoprenoid compound may be inserted into the cavities. As a result, the isoprenoid compound is released only in response to specific events, such as wiping or rubbing the substrate across the skin.

As mentioned, farnesol is derived from plant extracts. Thus, essentially any plant extracts from which farnesol or other effective isoprenoids can be derived can be applied to the skin-contacting surface of the article of the invention. Suitable plant extracts may include essential oils from orange blossom, rose, jasmine, linden flowers, as well as any other plants that contain farnesol. Other types of plant extracts from which isoprenoid compounds may be derived include Basil (Ocimum basilicum); Bay Laurel (Laurel nobilis); Bergamot (Citrus aurantium bergamia); Calendula; Cardamom (Elettaria cardamomum); Cedarwood (Cedrus atlantica); Citronella (Cymbopogon nardus); Chamomile, German (Matricaria recutita); Chamomile, Roman (Anthemis nobilis); Clove (Eugenia caryophyllata); Cypress (Cupressus sempervirens); Eucalyptus (Eucalyptus citriodora); Eucalyptus (Eucalyptus radiata); Frankincense (Boswellia carterii); Geranium (Pelargonium×asperum); Ginger (Zingiber officinale); Grapefruit Peel (Citrus×paradisi); Helichrysum (Helichrysum italicum); Juniper (Juniperus communis); Lavender (Lavendula angustifolia); Lavandin (Lavandin abrialis); Lemon Peel (Citrus limon); Lemongrass (Cymbopogon flexuosus citraliferum); Lime peel (Citrus aurantifolia); MQV (Melaleuca quinquenervia viridiflora) also known as Niaouli; Myrrh (Commiphora molmol); Neroli (Citrus aurantium, flowers); Orange (Citrus sinensis); Palmarosa (Cymbopogon martinii); Peppermint Mitcham (Mentha×piperita); Petitgrain (Citrus aurantium, leaves); Pine (Pinus sylvestris) also known as Scotch Pine; Ravensara (Ravensara aromatica); Rosehip (rosa rubiginosa); Rosemary (Rosemarinus officinalis camphor type); Rosemary (Rosemarinus officinalis verbenone type); Sea Buckthorn Berry; Tarragon (Artemisia dracunculus); Tea Tree (Melaleuca alternifolia); Thyme (Thymus vulgaris, linalool type); Thyme (Thymus vulgaris thymol type); Vetiver (Vetiveria zizanoides); Vitex leaf (Vitus agnus castus); Ylang Ylang (Cananga odorata).

The isoprenoid compounds that inhibit fungi attachment to skin can be delivered from the substrate to a wearer's skin using any of a number of different compositions. Examples of suitable vehicles include lotions, emulsions, creams, gels, aqueous vehicles, encapsulation, microencapsulation, and coating of nanoparticles. Examples of compositions that enhance and/or target the delivery of the isoprenoid compound to the wearer's skin, such as petrolatum, alcohols, glycerols, waxes, or other hydrophobic compounds. Alternatively, vehicles having various degrees of complexity may be used, ranging from simple vehicles made of a singular substance to emulsions to rather complex vehicles such as particulate materials bearing specific ligands to target the isoprenoid compound to particular locations within the skin environment.

One approach of using ligand-specific material involves targeting the user's skin. In one embodiment of the invention, the farnesol molecule is attached to specific ligands that have an affinity for the skin surface. These ligands include antibodies and lectins specific for the carbohydrate and protein domains in the skin. Specific ligands include: 5-chloro-7-iodoquinolin-8-ol, ethylenediaminetetraacetic acid, sodium diethyldithiocarbamate, L-histidine, the selectin family of ligands consisting of three members, namely E-, P-, and L-selectin, and heparin-binding ligands. Other lectins that can be used to target the skin include peanut lectin (PNA), soy lectins, wheat lectins (WGA), and aloe lectins.

Another approach of using ligand-specific material involves targeting the yeast. In one embodiment of the invention, the molecules on the cell wall or cell membrane of the yeast can be targeted. Specific molecules that can be targeted include N-acetyl galactosamide, N-acetyl glucosamide, and N-mannopyranoside. Lectins specific to these molecules work well, with examples including antibodies and plant lectins. As a more specific example, lectins from plants such as the jack bean plant work well. Some carbohydrates such as mannose can also be used to target skin ligands and yeast cell surface ligands. Furthermore, bacterial produced lectins from Pediococcus damnosus, Bacillus subtilis, Erwinia herbicola, Lactococcus sp., Micrococcus luteus, Proteus vulgaris, and Erythrina sp. can be used to target farnesol to the yeast.

Referring to FIG. 1, a diaper 10 is shown as an example of a personal care absorbent garment. Other personal care absorbent garments, such as diaper pants, training pants, absorbent underpants, swimwear, and incontinence garments, are each constructed in a manner similar to the diaper 10. More specifically, disposable absorbent garments 10 of this type generally include a liquid impermeable back sheet member 12, an absorbent assembly 16, and a liquid permeable bodyside liner 18. The bodyside liner 18 or a tissue material forms a skin-contacting surface that comes into contact with the wearer's skin. Typically, the back sheet member 12 is joined to the bodyside liner with the absorbent assembly 16 disposed between the back sheet member 12 and the bodyside liner 18.

Referring to FIG. 2, a feminine care pad 30 is shown as an example of a feminine care article. This feminine care pad 30, like other types of feminine care articles, includes an absorbent assembly 16 as a main component, with the absorbent assembly 16 forming a skin-contacting surface that comes into contact with the wearer's skin.

Referring to FIG. 3, a bandage 32 is shown as an example of a health care article. Other examples of health care articles include medical care articles, dental care articles, veterinary care articles, and all sorts of wound dressings. The bandage 32 includes an absorbent assembly 16 attached to an adhesive strip 34, with the absorbent assembly 16 forming a skin-contacting surface that comes into contact with the wearer's skin.

Referring to FIG. 4, an absorbent wipe 36 is shown as an example of a personal care article. Other examples of personal care articles include bath tissue, facial tissue, lotions, creams, and combinations of any of these. The absorbent wipe 36 includes an absorbent assembly 16 substrate that has been soaked in a cleansing solution. The entire surface area of the absorbent wipe 36 may be considered a skin-contacting surface that may come into contact with the wearer's skin.

Each of the embodiments of personal care articles shown in FIGS. 1-4 includes an absorbent assembly 16 of some sort. In general, the absorbent assembly 16 absorbs and retains bodily fluids, such as urine, menses, feces, pus, and other body exudates. The absorbent assembly 16 is suitably compressible, conformable, and non-irritating to the wearer's skin. The absorbent assembly 16 may include a wide variety of liquid absorbent materials commonly used in absorbent articles. Absorbent assemblies 16 typically include a porous fibrous matrix 22 and high absorbency material 24, as shown in FIG. 1.

The porous fibrous matrix 22 of the absorbent assembly 16 is suitably an air laid batt of fluff and high absorbency material 24 which may be formed in many ways, as known to those skilled in the art. The absorbent assembly 16 may include an air-formed mixture of high absorbency superabsorbent material 24 and fibers 22, suitably of fluff pulp. The mixing of the fluff fibers 22 and the high absorbency material 24 can be homogeneous, graduated, or layered. Also, fibers 22 other than fluff pulp, such as chemically stiffened and thermo-mechanical pulps, can be used.

In addition, the absorbent assembly 16 can include absorbent material other than air formed fluff 22 and superabsorbent material 24. For example, coform materials, known to those skilled in the art, can be used to make the absorbent as long as they also contain high absorbency materials. In addition, wet formed composite materials including a combination of fibers and high absorbency materials can also be used.

Stabilized air-laid materials including a mixture of fibers, binder fibers, and high absorbency materials which are bound together by latex binding or through-air bonding are also usable as absorbent materials. Additionally, any material known in the art that serves to absorb body exudates can be used to construct the absorbent assembly 16 as shown in the present invention.

The absorbent assembly 16 may also include a wrap layer 26 to help maintain the integrity of the fibrous absorbent assembly 16. This wrap layer 26 may include a cellulosic tissue or spunbond, meltblown, or bonded-carded web material composed of synthetic polymer filaments, such as polypropylene, polyethylene, polyesters, or the like, or natural polymer filaments such as rayon or cotton. The wrap layer 26 may be made of the same materials as those used in the bodyside liner 18 or be made of materials differing from those used in the bodyside liner 18. In some cases, the bodyside liner 18 may be absent, and the wrap layer 26, also referred to as tissue material 20, will serve as the bodyside layer 18 of the absorbent article 10, coming in contact with the wearer's skin.

The absorbent assembly 16 can include additional components to assist in the acquisition, distribution, and storage of bodily exudates, such as a dusting layer, a transport layer, a wicking or acquisition/distribution layer, an intake layer, or a surge layer.

The bodyside liner 18 includes a nonwoven or other soft material for contacting the wearer's skin. The bodyside liner 18 is compliant and soft feeling to the wearer. The bodyside liner 18 may be any soft, flexible, porous sheet that is aqueous liquid permeable, permitting aqueous liquids to readily penetrate into its thickness. A suitable bodyside liner 18 may be manufactured from a wide range of materials, such as natural fibers (e.g., wood or cotton fibers), synthetic fibers (e.g., polyester or polypropylene fibers) or from a combination of natural and synthetic fibers or reticulated foams and apertured plastic films.

The bodyside liner 18 is formed of an aqueous liquid permeable material so that aqueous liquid waste, and possibly semi-solid waste as well, can pass through to the absorbent assembly 16 and be absorbed by the absorbent assembly 16 of the absorbent article 10. A suitable bodyside liner 18 may include a nonwoven web, a spunbond, meltblown or bonded-carded web including synthetic polymer filaments or fibers, such as polypropylene, polyethylene, polyesters or the like, a perforated film, or a web or natural polymer filaments or fibers such as rayon or cotton.

The back sheet member 12 is needed to prevent aqueous liquid strike through to the outer clothing when bodily fluid is discharged onto the absorbent assembly 16 of the absorbent article 10. The back sheet member 12 typically includes an aqueous liquid impermeable film such as polyethylene, but may alternatively be an aqueous liquid permeable material. In construction of the disposable absorbent article 10, the back sheet member 12, acting as a barrier, should retard the movement of the aqueous liquid through the absorbent article 10 by making the back sheet member 12 resistant to penetration normally encountered under wearing conditions. The back sheet member 12 desirably includes a material that is formed or treated to be aqueous liquid impermeable.

The absorbent articles 10 may include various other features, such as elastic members, fastening systems, and barrier structures, as known to those skilled in the art.

### EXAMPLE

The following Example demonstrates the inhibition of yeast attachment caused by farnesol. The Example utilized both tape striped volar forearm skin and cyanoacrylate pulls of volar forearm skin. A description of the protocol and results are given below.

Tape Strips vs. Cyanoacrylate Skin Pulls

The first step in developing anti-adherence treatments is the development of a high fidelity model to measure adherence of yeast to the skin. Development of such a model requires an appropriate surface on which to measure the attachment. With regard to this, the most appropriate substrate is human skin and/or mucous membranes. Two easily performed methods to collect human skin are tape strip and cyanoacrylate skin pulls.

The cyanoacrylate glue skin pull technique appeared to be the best method for removing a continuous layer of unadulterated skin from the human forearm. The tape pull technique was effective for the removal of skin from the human arm but silicone appears to contaminate the surface of the collected skin. The silicone arises from the adhesive of the tape and is presumably transferred through the skin or around fractured skin to the surface of the pull. Furthermore, the tape pull method results in a discontinuous sheet of skin that requires subsequent blocking of the tape adhesive with bovine serum albumin. This procedure has the potential for producing areas of the sample to which the yeast could bind to that are not human skin.

The sample that most closely mimics real skin is the best to use for screening of novel anti-adherent technologies. Cyanoacrylate pulls would appear to most closely mimic the surface of the skin. Therefore, the results obtained using the skin pull are the most representative of native conditions.

Organism

Candida albicans (ATCC 10231) was used in this study. C. albicans was sub-cultured onto a Sabourads medium fortified with glucose (SAB-Dex) agar plate (Becton Dickinson, Cockeysville, Md.) overnight at 37 degrees Celsius. The following day, 2-3 isolated C. albicans colonies were inoculated into 20 ml SAB-Dex broth and incubated overnight shaken at 220 rpm at 32 degrees Celsius for 18 hours. The broth culture was diluted to 1×10⁵ CFU/ml with phosphate buffer (VWR Industries, Batavia, Ill.).

Visual Release Protocol

The following protocol was used to measure the inhibition of C. albicans to skin (skin tape strips and cyanoacrylate skin pulls) by farnesol. Skin tape strips were made by pulling D-Squame skin sampling discs (CuDerm Corporation, Dallas, Tex.) four times from adjacent adult male volar forearm sites. Cyanoacrylate pulls were done by putting a small amount of super glue on the skin then placing a glass slide on the wetted surface. The glue was allowed to dry then the slide was removed, pulling off the stratum corneum. The tape strips and glue pulls (both will be referred to as strips in subsequent descriptions) were placed into deep six-well plates (Becton Dickinson, Franklin Lakes, N.J.). The strips were then blocked with 2.0 ml of 5% Bovine Serum Albumin (BSA) (Sigma, St. Louis, Mo.) in PBS buffer (150 mM NaCl, 50 mM Potassium Phosphate (KP), pH 7.4) for 60 minutes at 33 degrees Celsius while shaking at 220 rpm.

Next, each well's fluid was removed and 1.0 ml (10⁵ Colony Forming Units (CFU)/ml) C. albicans was added to each strip. Then, 1.0 ml of Trypticase Soy Broth (TSB) [Difco Labs, Detroit, Mich.] containing 2% farnesol (DRAGOCO, Totowa, N.J.) was added to each strip, and the plates incubated at 33 degrees Celsius for 60 minutes. The fluid was aspirated, and the strips were washed 3 times with 3.0 ml PBS. Both sides of each tape strip were washed with a stream of PBS then placed in fresh 6-well plates.

Each strip was fixed by adding 2.0 ml of 2.5% Glutaraldehyde (Sigma Chemical, St. Louis, Mo.) to each of the wells of the 6-well plates for 10 minutes. The tape strips were then washed 3 times with 3.0 ml distilled water and stained by adding 0.5 ml Calcofluor White (Difco, Ann Arbor, Mich.) to the wells for 10 to 15 minutes. The tape strips were again washed 3 times with distilled water and then flooded with 2 ng/ml Nile Red (Sigma Chemical, St. Louis, Mo.) and allowed to air dry.

Once the tape strips air-dried, the yeast cells were enumerated automatically utilizing a Nikon Eclipse TE 300 fluorescent microscope fitted with a DAPI excitation filter and a Triple Pass barrier filter (Tokyo, Japan). The counting procedure was automated (Table 1 and Table 2) through the use of MetaMorph (Universal Imaging, Dowingtown, Pa.) software such that each sample had 30 views. Each image view was a 6000 um². The total field of view was approximately 25% of the total tape strip and 75% of the cyanoacrylate pull. The percent inhibition was calculated as follows: 100-((sample # of cells)/(control # of cells)×100). Approximately 10⁴ yeast cells bound to a 22-mm diameter D-Squame tape strip under these conditions.

TABLE 1
Image Capture Parameters for Both Types of Skin Pulls

| Mode | Multi-Dimension Capture |
|---|---|
| Illumination | 100% DAPI 385-415 nm |
| Barrier Filter | Multi-Pass 50-470 nm, 510-540 nm, and 590-650 nm |
| Objective | 10 x |
| View Size | 6000 um²/plane (90 um × 67.2 um) |
| Exposure | 150 ms |
| Auto-Scale | Auto-Scale ON (high = 0.05) |

TABLE 2
Image Analysis Parameters for Both Types of Skin Pulls

| | Tape Pull | Cyanoacrylate Pull |
|---|---|---|
| Area Filter (pixels) | 400 | 250 |
| Threshold | 2791-4095 | 2791-4095 |
| Auto-Scale | 2000-4095 | 2000-4095 |
| Standard Area Size (pixels) | 25 | 25 |

Results

It was found that farnesol inhibited 94% of the yeast from attaching to cyanoacrylate pulled skin and 64% of the yeast to tape strip pulled skin (Table 3). The numbers of cell count on the treated samples were all statistically different from nontreated samples (Table 4). This data implies that farnesol can inhibit the attachment of yeast to skin.

TABLE 3
Summary of Results for Both Types of Skin Pulls (view = 6000 um²)

| | Tape Strip | | Cyanoacrylate Pull | |
|---|---|---|---|---|
| | Control | Farnesol | Control | Farnesol |
| 90 View Total | 19251 | 6963 | 15322 | 955 |
| AVG per view | 213.9 | 77.4 | 168.4 | 10.6 |
| SD per view | 111.2 | 61.8 | 117.3 | 10.6 |
| N | 90 | 90 | 91 | 90 |
| % Inhibition | | 63.8 | | 93.8 |

TABLE 4
Summary of Statistics (Toukai-Kramer Test)
for Both Types of Skin Pulls

P value

| | Tape Strip | Cyanoacrylate Pull |
|---|---|---|
| Control vs. Farnesol | *** P<0.001 | *** P<0.001 |

It will be appreciated that details of the foregoing embodiments, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

What is claimed is:

1. A personal care article, comprising:
a personal care article substrate; and
an isoprenoid compound applied to at least a skin-contacting surface of the substrate in an amount effective to inhibit pathogenic fungi attachment to skin.

2. The personal care article of claim 1, wherein the isoprenoid compound comprises at least one of the group consisting of farnesol, atlantol, cedrol, (−)alpha-bisabolol spathulenol, citronellol, geraniol, borneol, cedrol:borneol, trans-pinocarveol, and combinations thereof.

3. The personal care article of claim 1, wherein the substrate comprises at least one of the group consisting of a personal care absorbent garment, a feminine care article, a health care article, a pre-moistened wipe, an absorbent wipe, bath tissue, facial tissue, a lotion, and a cream.

4. The personal care article of claim 1, wherein the substrate comprises a nonwoven web.

5. The personal care article of claim 1, further comprising a composition selected from the group consisting of petrolatum, alcohols, glycerols, waxes, hydrophobic compounds, and combinations thereof.

6. The personal care article of claim 1, wherein the isoprenoid compound is incorporated within a vehicle selected from the group consisting of lotions, emulsions, creams, gels, aqueous vehicles, encapsulation, microencapsulation, and coating of nanoparticles.

7. The personal care article of claim 1, wherein the pathogenic fungi comprises at least one of the group consisting of Candida albicans, Mallessia, Tricophyton, Epidermophyton, Scytalidium, Fusarium, Acremonium, Aspergillus, Scopulariopsis, and Pityrosporum.

8. The personal care article of claim 1, wherein the isoprenoid compound is present in solution at a concentration of between about 0.001% and about 2% by weight of the solution.

9. The personal care article of claim 1, wherein the isoprenoid compound is present in solution at a concentration of between about 0.001% and about 0.1% by weight of the solution.

10. The personal care article of claim 1, wherein the isoprenoid compound is present in solution at a concentration of between about 0.001% and about 0.01% by weight of the solution.

11. A personal care article, comprising:
a personal care article substrate; and
farnesol applied to at least a skin-contacting surface of the substrate in an amount effective to inhibit pathogenic yeast attachment to skin.

12. The personal care article of claim 11, wherein the substrate comprises a personal care absorbent garment selected from the group consisting of a diaper, a diaper pant, a training pant, an absorbent underpant, swimwear, and an incontinence garment.

13. The personal care article of claim 11, wherein the substrate comprises a feminine care article.

14. The personal care article of claim 11, wherein the substrate comprises a health care article.

15. The personal care article of claim 11, wherein the substrate comprises at least one of the group consisting of a pre-moistened wipe, an absorbent wipe, bath tissue, facial tissue, a lotion, and a cream.

16. The personal care article of claim 11, wherein the substrate comprises a nonwoven web.

17. The personal care article of claim 11, further comprising a composition selected from the group consisting of petrolatum, alcohols, glycerols, waxes, hydrophobic compounds, and combinations thereof.

18. The personal care article of claim 11, wherein the farnesol is incorporated within a vehicle selected from the group consisting of lotions, emulsions, creams, gels, aqueous vehicles, encapsulation, microencapsulation, and coating of nanoparticles.

19. The personal care article of claim 11, wherein the pathogenic yeast comprises Candida albicans.

20. The personal care article of claim 11, wherein the substrate comprises a plurality of cavities and the farnesol resides within at least some of the plurality of cavities.

21. The personal care article of claim 11, wherein the farnesol is present in solution at a concentration of between about 0.001% and about 2% by weight of the solution.

22. The personal care article of claim 11, wherein the farnesol is present in solution at a concentration of between about 0.001% and about 0.1% by weight of the solution.

23. The personal care article of claim 11, wherein the farnesol is present in solution at a concentration of between about 0.001% and about 0.01% by weight of the solution.

24. A method of inhibiting pathogenic fungal attachment to skin, comprising:
applying an effective amount of an isoprenoid compound to at least a skin-contacting surface of a personal care article.

25. The method of claim 24, wherein the isoprenoid compound comprises at least one of the group consisting of farnesol, atlantol, cedrol, (−)alpha-bisabolol spathulenol, citronellol, geraniol, borneol, cedrol:borneol, trans-pinocarveol, and combinations thereof.

26. The method of claim 24, wherein the personal care article comprises at least one of the group consisting of a personal care absorbent garment, a feminine care article, a health care article, a pre-moistened wipe, an absorbent wipe, bath tissue, facial tissue, a lotion, and a cream.

27. The method of claim 24, wherein the skin-contacting surface of the personal care article comprises a nonwoven web.

28. The method of claim 24, wherein the pathogenic fungi comprises at least one of the group consisting of Candida albicans, Mallessia, Tricophyton, Epidermophyton, Scytalidium, Fusarium, Acremonium, Aspergillus, Scopulariopsis, and Pityrosporum.

29. The method of claim 24, further comprising a composition selected from the group consisting of petrolatum, alcohols, glycerols, waxes, hydrophobic compounds, and combinations thereof.

30. The method of claim 24, wherein the isoprenoid compound is applied to the substrate in a vehicle selected from the group consisting of lotions, emulsions, creams, gels, aqueous vehicles, encapsulation, microencapsulation, and coating of nanoparticles.

31. The method of claim 24, wherein the isoprenoid compound is applied to the personal care article as a sprayed-on-additive.

32. The method of claim 24, wherein the isoprenoid compound is applied to the personal care article by incorporating the isoprenoid compound into a melt from which at least the skin-contacting surface of the personal care article is made.

33. The method of claim 24, wherein the isoprenoid compound is applied to the personal care article by soaking the personal care article in a solution including the isoprenoid compound.

34. The method of claim 24, wherein the skin-contacting surface of the personal care article comprises a plurality of cavities and the isoprenoid compound is inserted into at least some of the plurality of cavities.

35. The method of claim 24, further comprising using ligand-specific material to deliver the isoprenoid compound to a user's skin.

36. The method of claim 24, wherein the isoprenoid compound is applied to the personal care article in solution at a concentration of between about 0.001% and about 2% by weight of the solution.

37. The method of claim 24, wherein the isoprenoid compound is applied to the personal care article in solution at a concentration of between about 0.001% and about 0.1% by weight of the solution.

38. The method of claim 24, wherein the isoprenoid compound is applied to the personal care article in solution at a concentration of between about 0.001% and about 0.01% by weight of the solution.

39. A method of inhibiting pathogenic yeast attachment to skin, comprising:
applying an effective amount of farnesol to at least a skin-contacting surface of a personal care article.

40. The method of claim 39, wherein the skin-contacting surface of the personal care article comprises a nonwoven web.

41. The method of claim 39, wherein the personal care article comprises a personal care absorbent garment selected from the group consisting of a diaper, a diaper pant, a training pant, an absorbent underpant, swimwear, and an incontinence garment.

42. The method of claim 39, wherein the personal care article comprises a feminine care article.

43. The method of claim 39, wherein the personal care article comprises a health care article.

44. The method of claim 39, wherein the personal care article comprises at least one of the group consisting of a pre-moistened wipe, an absorbent wipe, bath tissue, facial tissue, a lotion, and a cream.

45. The method of claim 39, wherein the pathogenic yeast comprises Candida albicans.

46. The method of claim 39, further comprising a composition selected from the group consisting of petrolatum, alcohols, glycerols, waxes, hydrophobic compounds, and combinations thereof.

47. The method of claim 39, wherein the farnesol is applied to the substrate in a vehicle selected from the group consisting of lotions, emulsions, creams, gels, aqueous vehicles, encapsulation, microencapsulation, and coating of nanoparticles.

48. The method of claim 39, wherein the farnesol is applied to the personal care article as a sprayed-on-additive.

49. The method of claim 39, wherein the farnesol is applied to the personal care article by incorporating the farnesol into a melt from which at least the skin-contacting surface of the personal care article is made.

50. The method of claim 39, wherein the farnesol is applied to the personal care article by soaking the personal care article in a solution including the farnesol.

51. The method of claim 39, wherein the skin-contacting surface of the personal care article comprises a plurality of cavities and the farnesol is inserted into at least some of the plurality of cavities.

52. The method of claim 39, further comprising using ligand-specific material to deliver the farnesol to a user's skin.

53. The method of claim 39, wherein a plant extract comprising the farnesol is applied to at least the skin-contacting surface of the personal care article.

54. The method of claim 53, wherein the plant extract comprises essential oils from at least one of the group consisting of orange blossom, rose, jasmine, linden, and combinations thereof.

55. The method of claim 39, where in the farnesol is applied to the personal care article in solution at a concentration of between about 0.001% and about 2% by weight of the solution.

56. The method of claim 39, wherein the farnesol is applied to the personal care article in solution at a concentration of between about 0.001% and about 0.1% by weight of the solution.

57. The method of claim 39, wherein the farnesol is applied to the personal care article in solution at a concentration of between about 0.001% and about 0.01% by weight of the solution.
